(19) United States
(12) Patent Application Publication
(10) Pub. No.: US 2004/0242956 A1
(43) Pub. Date: Dec. 2, 2004

(54) System for controlling fluid in a body

(76) Inventor: Sean K. Scorvo, Bend, OR (US)

Correspondence Address: Kevin L. Russell, Suite 1600, 601 SW Second Ave., Portland, OR, 97204-3157, US

(21) Appl. No.: 10/630,579

(22) Filed: Jul. 29, 2003

Related U.S. Application Data
(60) Provisional application No. 60/399,585, filed on Jul. 29, 2002.

(51) Int. Cl. A61F 2/02

(52) U.S. Cl. 600/30; 600/38

(57) ABSTRACT

A system for controlling the flow of fluid in a body includes an implantable device for controlling the flow of fluid in a duct, a controller, and, if appropriate, a pressure assist device for a reservoir that is a source of fluid flowing in the duct.

### CROSS-REFERENCE TO RELATED APPLICATIONS

This application claims the benefit of U.S. Provisional Application No. 60/399,585 filed Jul. 29, 2002.

### BACKGROUND OF THE INVENTION

The present invention relates to medical devices and, more particularly, to a system, including implantable medical devices, for controlling fluid in a body.

Fluid control within the human body is important to a number of functions. For examples, urinary incontinence, urinary retention, and male impotence are disorders affecting significant segments of the population that involve the control of fluid in the body.

Urinary incontinence is a dysfunction of the bladder to securely retain urine until the individual intends to void. Urinary incontinence effects between 1-1.5% of men and 10-30% of women between the ages of 15 and 64 years. In 1996, it was estimated that 25 million Americans were affected by urinary incontinence with an annual cost of about $16.4 billion. There are many underlying causes of urinary incontinence, including, but not limited too, muscular dystrophy, cerebral palsy, nervous system disorders, brain damage, anatomic changes associated with previous pregnancy, aging induced changes in the relationship of the bladder to the urethra, side effects of medication, and spinal cord injuries.

On the other hand, urinary retention, the inability to empty the bladder during urination, also effects a significant segment of the population. Urinary retention typically affects those with congenital or accidental neural cord deficits, neurologic disorder, or prostatic hypertrophy which obstructs the urethra. Neural cord or neurologic deficit can adversely effect the signal from the stretch receptors in the bladder wall detecting bladder pressure to the smooth muscles that contract to compress the bladder during urination. Urinary retention can lead to urinary incontinence. If urine is retained in the bladder, the pressure may increase to a point where urine is forced back into the kidneys or out through the urethra by minor bladder contractions.

The principal elements of the urinary system are the kidneys, the ureters, the urinary bladder and the urethra. The kidneys selectively remove water and soluble salts produced during metabolism; including urea, uric acid, and creatinine, from the blood stream. The ureters are thick walled, muscular tubes that connect the kidneys to the urinary bladder. Muscle fibers of the ureters exert a clamping (sphincter) effect that is modulated so that waves of muscular contractions pass downward along the ureter and urine passes into the bladder in small, periodic spurts, rather than as a steady stream. The urinary bladder is a muscular reservoir that serves as temporary storage of urine received from the kidneys and discharged at intervals through the urethra. The urethra is a narrow passageway through which urine flows from the bladder to the outside. The flow of urine in the urethra is controlled by the urethral or outflow sphincter, a muscular region that surrounds a portion of the urethra and acts to constrict the urethra to block flow.

Urinary continence requires closure of the urethra and relaxation of the bladder during the urine collection phase and simultaneous relaxation of the sphincter muscles of the urethra and an involuntary contraction of the detrusor muscles in the bladder wall to generate an intravesical pressure greater than the inlet pressure at the urethral sphincter during urination. These functions of the urethra and bladder are centrally coordinated and non-separable. The sensation of urge is mediated during bladder filling by slowly adapting stretch receptors in the bladder wall. The stretch receptors provide the triggering signal for relaxation of the urethral sphincter and activation of the detrusor muscle that supplies the force for a sustained bladder contraction. Where the urethral sphincter muscles, the detrusor muscles, or their associated nerves and fibers become inoperative because of disease, damage, or otherwise, the problem of involuntary escape of urine (incontinence) or the inability to empty the bladder (urinary retention) results.

Incontinence is treated in several ways, including surgery, behavior modification, drugs to inhibit bladder contractions, and devices to capture discharges, such as adult diapers. Drugs may have unwanted side effects, such as urinary retention, low blood pressure, constipation, abdominal cramping, and blurred vision. Sacral nerve stimulators surgically implanted in the abdomen are an alternative treatment, but only efficacious for a limited number of patients. The urethra may be surgically banded by wrapping another muscle around the urethra or injecting collagen but these treatments can be surgically complex and may be overly constricting leading to urinary retention.

An artificial sphincter is one method of treating urinary incontinence. A sphincter is a muscle typically encircling a fluid duct, such as the urethra, so that contraction of the muscle will constrict the passageway and occlude the flow of fluid in the duct. Most artificial sphincters are circular cuff members that intermittently occlude the urethra or other bodily duct in response to fluid pressure in the cuff member. Early attempts to prevent male incontinence involved externally clamping the penis. Examples of exteriorly applied artificial sphincter mechanisms for controlling incontinence are illustrated in U.S. Pat. Nos. 2,455,859 and 2,533,924 issued to F. E. Foley on Dec. 7, 1948 and Dec. 12, 1950, respectively. However, exterior pressure sufficient to stop urinary flow tends to compromise circulation causing pain, skin alteration, and thrombosis. An analogous artificial sphincter for women that compresses the urethra between the vaginal wall and the pubic bone shares these disadvantages.

Although not without certain disadvantages, implantable artificial sphincters represent a significant improvement over earlier external clamping techniques and implantable artificial sphincters have been disclosed in the prior art. Helms et al, U.S. Pat. No. 4,256,093, teaches the use of a fluid filled urethral collar which is contracted by manually squeezing a bulb implanted in the scrotum. U.S. Pat. No. 3,815,576 issued to Donald R. Balaban teaches the use of a fluid filled flexible container implanted in the patient which is squeezed manually to actuate a piston-cylinder in a U-shaped clamp. Typically, these implanted prior art devices require a bulbous pump to be implanted in the scrotum of the male or in the labium of the female. In order to initiate urine flow the patient must actuate the pump making the device psychologically and cosmetically undesirable.

Artificial sphincter cuff pressure above 40 cm of water produces necrosis (tissue death) in the urethra. However, voluntary or involuntary tensing of the diaphragm or abdominal wall due to walking, sitting, coughing, or laughing can produce high pressure transients in the bladder that may cause an artificial sphincter to leak. McWhorter et al, U.S. Pat. No. 3,744,063, teaches controlling the flow of a fluid into the sphincter to control pressure exerted by an artificial sphincter. However, pressure is increased by manually actuating an implanted pump chamber when the patient detects dripping incontinence and it is unlikely that the patient would be able to successfully respond to the rapid changes in bladder pressure accompanying normal events. In addition, the urethra tissue swells immediately after surgery and, to avoid tissue necrosis, the artificial sphincter is typically left unfilled until the swelling subsides. Successful implantation of a fluid filled, artificial sphincter typically requires providing an exterior means of filling the sphincter or performing a second operation to fill the sphincter.

Sayet et al., U.S. Pat. No. 6,319,191 B1, discloses an implantable fluid flow control device or artificial sphincter comprising a cylindrical shell for encircling the fluid duct and a piston driven plunger that compresses the fluid duct against an interior surface of the cylindrical shell. The piston can be driven hydraulically or pneumatically or, preferably, by an electromechanical solenoid. While the electromechanical solenoid avoids many of the cosmetic and psychological problems associated with implantation of a hydraulic or pneumatic power source, the switch-actuated solenoid is a bi-state device and the artificial sphincter is not responsive to pressure transients which can produce excessive pressure, incontinence, and tissue damage. Miller, U.S. Pat. No. 5,509,888, discloses a device and method for regulating fluid flow within the body that includes a magnetorheological fluid actuator in a surgically implantable collar. A programmable control device outputs signals to alter magnetic fields to change the density and viscosity of the magnetorheologic fluid in the actuator and cause the actuator to expand within the collar to occlude the fluid duct.

Urinary retention is treated by surgical removal or treatment of the urethral obstruction. Prostatic laser surgery, microwave treatment, and transurethral resection of the prostate may alleviate the obstruction. Drugs may also be used to treat benign prostatic hypertrophy by either blocking further hypertrophy or inhibiting smooth muscle contraction around the prostatic urethra to enhance the flow of urine. However, the surgical treatments are not without risk including bleeding, loss of sexual function and infection and the medicinal treatments are not universally effective and may produce side effects. Acute urinary retention such as may result from a loss of neurologic function secondary to paralysis or inflamation, such as prostatitis, is often treated with a catheter than is implanted by way of the urethra to facilitate the flow of urine. Likewise, long term urinary incontinence or retention may be treated with a catheter (known as an indwelling Foley catheter). However, use of a urinary catheter is associated with trauma resulting from placement of the device, significantly increased risk of infection, and psychological trauma.

Male impotence is often caused inadequate control of fluid in a bodily duct, more specifically, decreased vasoconstriction of the superficial and/or deep dorsal veins of the penis. Treatments for male impotence includes a number of penile implants all of which invoke psychological and physical trauma. Miller, U.S. Pat. No. 5,509,888, discloses the use of an artificial sphincter actuated by a magnetorheologic fluid to control blood flow in the superficial and/or deep dorsal veins of the penis to treat impotence.

Artificial fluid control valves for bodily functions are a continuing source of problems for the medical industry. The tissues around sphincters are not tolerant to prolonged blood deprivation and are sensitive to forces applied to control fluid flow and prolonged insertion of catheters to relieve blockage can easily lead to infection. What is desired, therefore, is an implantable system for controlling the flow of fluid in a bodily duct that is adaptable and physically and psychologically acceptable.

### BRIEF DESCRIPTION OF THE DRAWINGS

FIG. 1A is a perspective view of an implantable fluid flow control device in a flow enabling state.

FIG. 1B is a perspective view of the implantable fluid flow control device of FIG. 1A in a fluid blocking state.

FIG. 1C is a sectional view of the implantable fluid flow control device of FIG. 1A.

FIG. 1D is a sectional view of the implantable fluid flow control device of FIG. 1C taken along line 1-1.

FIG. 2A is an upper front perspective view of an electroactive polymer transducer.

FIG. 2B is an upper front perspective view of the electroactive polymer transducer of FIG. 8A in an actuated state.

FIG. 3A is a front elevation of a second embodiment of an implantable fluid flow control device in a flow enabling state.

FIG. 3B is a sectional view of the implantable fluid flow control device of FIG. 3A in a flow blocking state.

FIG. 4A is an end view of a third embodiment of an implantable fluid flow control device in a flow enabling state.

FIG. 4B is an end view of the implantable fluid flow control device of FIG. 4A in a flow blocking state.

FIG. 4C is an end view of an alternative construction of the implantable fluid flow control device of FIG. 4A.

FIG. 5A is a cross-sectional view of a dilating element fluid flow control device implanted in a urethra in a flow blocking state.

FIG. 5B is a cross-sectional view of the dilating element fluid flow control device of FIG. 5A in a flow enabling state.

FIG. 6A is a cross-sectional view of a dilating element fluid flow control device of alternative construction implanted in a urethra in a flow blocking state.

FIG. 6B is a cross-sectional view of the dilating element fluid flow control device of FIG. 6A in a flow enabling state.

FIG. 7A is a cross-sectional view of an additional embodiment of an interiorly implantable fluid flow control device installed in a urethra in a flow blocking state.

FIG. 7B is a cross-sectional view of the additional embodiment of an interiorly implantable fluid flow control device of FIG. 7A in a flow enabling state.

FIG. 8 is a schematic illustration of a fluid pressure assist device encircling the exterior of a bladder.

FIG. 9 is a schematic illustration of a mesh useful for constructing the fluid pressure assist device of FIG. 8.

FIG. 10 is schematic illustration of an additional embodiment of a fluid pressure assist device encircling the exterior of a bladder

FIG. 11 is a schematic illustration of another embodiment of a fluid pressure assist device installed in the interior of a bladder.

FIG. 12 is a block diagram of an implantable fluid flow control system.

FIG. 13 is an illustration of a fluid control system utilized to treat male impotence.

FIG. 14A is a sectional view of an implantable fluid flow control device including a piezoelectric actuator in a flow enabling state.

FIG. 14B is a sectional view of the implantable fluid flow control device of FIG. 14A in a flow blocking state.

FIG. 15A is an elevation view of a second embodiment of an implantable fluid flow control device including a piezoelectric actuator in a flow enabling state.

FIG. 15B is an elevation view of the implantable fluid flow control device of FIG. 15A in a flow blocking state.

FIG. 16A is a sectional view of an interiorly implantable fluid flow control device including a piezoelectric actuator in a flow enabling state.

FIG. 16B is a sectional view of the implantable fluid flow control device of FIG. 16A in a flow blocking state.

FIG. 17A is a sectional view of a second embodiment of an interiorly implantable fluid flow control device including a piezoelectric actuator in a flow blocking state.

FIG. 17B is a sectional view of the implantable fluid flow control device of FIG. 17A in a flow enabling state.

FIG. 18 is an elevation view of a polymer-metal composite transducer.

FIG. 19A is a schematic cross-section of an implantable artificial sphincter including a polymer-metal composite transducer.

FIG. 19B is a schematic cross-section of the implantable artificial sphincter of FIG. 19A in a flow blocking state.

### DETAILED DESCRIPTION OF THE INVENTION

Controlling the flow of fluid in ducts of the human body is important to a number of functions. For examples, urinary incontinence, urinary retention, and male impotence are disorders affecting significant segments of the population that involve the control of fluid in the body. Typically, the flow of fluid in a duct is controlled by a sphincter, an annular muscle that surrounds the duct and contracts to occlude the duct blocking fluid flow or relaxes to open the duct enabling flow. Sufficient pressure differential to overcome the flow losses in the open duct may be provided by a muscular contraction of a fluid reservoir, such as the bladder, that is the source of the fluid in the duct. If the muscles of the sphincter or the muscles that compress the fluid reservoir become dysfunctional, the ability to control the flow of fluid is impaired. The implantable system for controlling the flow of fluid in a body includes implantable devices to control the pressure and flow of fluid in a duct and a control system to control the operation of these devices.

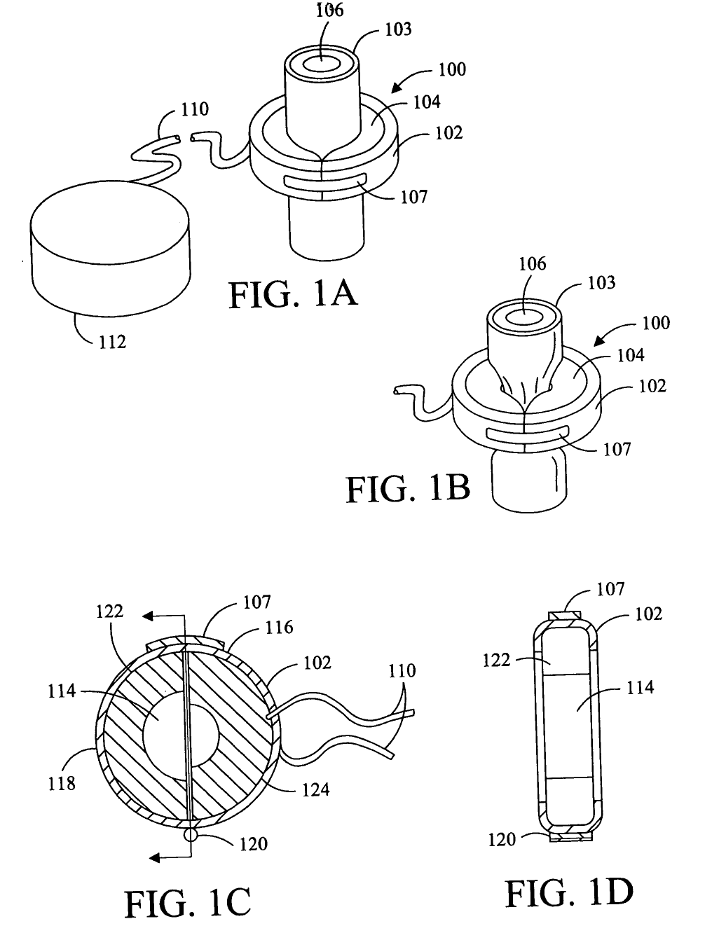

Referring in detail to the drawings wherein similar parts of the invention are identified by like reference numerals and, more specifically, to FIGS. 1A and 1B, a first embodiment of an implantable device for controlling a flow of fluid in a body duct or artificial sphincter 100 controls the flow of fluid in the duct or passageway 103 by selectively displacing a portion of the exterior of the duct's wall to occlude the aperture 106 formed by the inner surface of the duct wall. FIG. 1A shows the fluid flow control device 100 in a relaxed, non-constricting (flow enabling) state and FIG. 1B shows the same device in a constricting, flow blocking state. The artificial sphincter 100 is generally an annular cylinder having an inner aperture 114 adapted to substantially encircle the duct 103 and comprises, generally, a substantially annular occluding transducer 104 and a case 102 to restrain the transducer proximate to the duct and to anchor the reaction forces when the transducer is constricting the duct. The dimension of the inner aperture 114 is determined by the diameter of the fluid duct 103 to which the flow controlling device 100 is to be applied. For example, when used as a urinary incontinence treatment, the inner aperture 114 is sized to fit the urethra.

As illustrated, the artificial sphincter 100 is substantially circular, but may be constructed in other shapes to best fit the particular duct to which the device is to be applied. To avoid the necessity of severing the duct 103 during installation of an artificial sphincter 100 encircling the exterior surface of the duct, the artificial sphincter is split into substantially semicircular elements. As illustrated in FIG. 1C, the case 102 comprises two substantially semicircular elements 116, 118 joined at one end by a hinge 120. The opposite ends of the case elements 116, 118 are joinable by a closure device 107 that selectively secures the ends of the semicircular case elements when they are in position around the duct 103.

The case 102 may be manufactured from a formable material that is acceptable for implantation in the body. Exemplary materials include: titanium or titanium alloys, such as Ti-6Al4V; cobalt-based ferrous alloys; nickel alloys, such as nickel-titanium alloys, including NITINOL (which is an alloy of nickel (Ni) and titanium (Ti) developed by the Naval Ordinance Laboratories (NOL) at Silver Spring, Md., commercially available from Raytheon, Menlo Park, Calif.); ceramic materials, such as high-density aluminum oxide; carbon compounds such as pyrolytic carbon, vitreous carbon, or vapor deposited carbon on substrates; and plastic materials, such as medical grades of polyethylene, polypropylene, perfluorinated polymers, acrylic polymers, polyurethanes, or silicone rubbers.

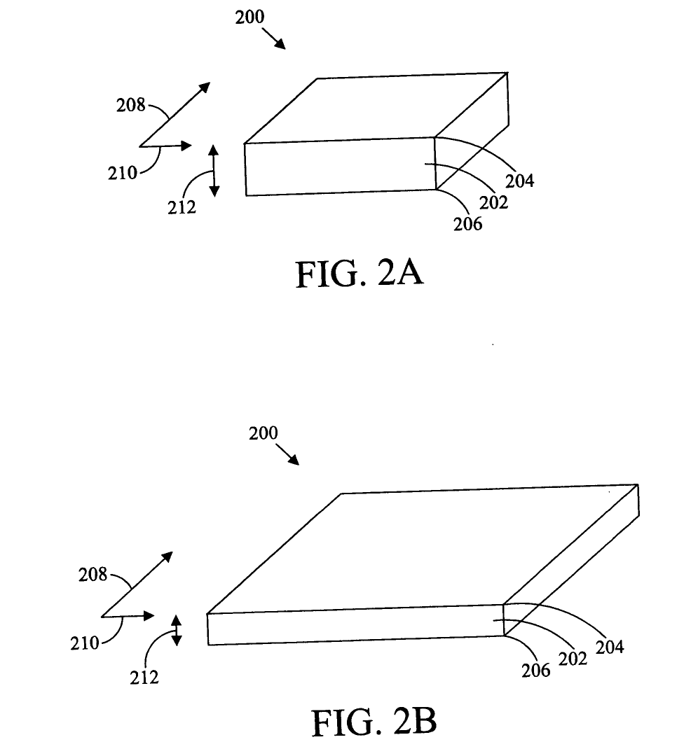

The occluding transducer 104 comprises an electroactive polymer actuator that deflects when electrical energy is applied. To help illustrate the performance of an electroactive polymer in converting electrical energy to mechanical energy, FIG. 2A illustrates a top perspective view of a transducer portion 200 comprising an electroactive polymer 202 for converting electrical energy to mechanical energy or vice versa. An electroactive polymer refers to a polymer that acts as an insulating dielectric between two electrodes and deflects upon application of a voltage differential between the electrodes. Top and bottom electrodes 204 and 206 are attached to the electroactive polymer 202 on its top and bottom surfaces, respectively, to provide a voltage difference across a portion of the polymer. The polymer 202 deflects with a change in electric field provided by the top and bottom electrodes 204 and 206. Deflection of the transducer portion 202 in response to a change in the electric field is referred to as actuation. As the polymer 202 changes in size, the deflection may be used to produce mechanical work. In general, deflection refers to any displacement, expansion, contraction, torsion, linear or area strain, or any other deformation of a portion of the polymer. The change in the electric field corresponding to the voltage difference applied to or by the electrodes 204 and 206 produces mechanical pressure within the polymer 202. In general, the transducer portion 200 continues to deflect until mechanical forces balance the electrostatic forces driving the deflection. The mechanical forces include elastic restoring forces of the polymer material, the compliance of the electrodes 204 and 206, and any external resistance provided by the load coupled to the transducer element.

Electroactive polymers and electroactive polymer transducers are not limited to any particular shape, geometry, or type of deflection. For example, a polymer and associated electrodes may be formed into any geometry or shape including tubes and rolls, stretched polymers attached between multiple rigid structures, and stretched polymers attached across a frame of any geometry, including curved or complex geometries; or a frame having one or more joints.

The occluding transducer 104 of the artificial sphincter 100 comprises two substantially semicircular elements 122 and 124 comprising an electroactive polymer. One of the elements 122, 124 is retained within each of the elements 116, 118 of the case 102 so that when the case is pivoted open for installation, the transducer elements are also separated. When the case 102 is closed and latched following installation, the elements 122, 124 of the transducer 104 are disposed on opposing sides of the duct 103 and substantially encircle the duct.

Materials suitable for use as an electroactive polymer may include any substantially insulating polymer or rubber (or combination thereof) that deforms in response to an electrostatic force or whose deformation results in a change in electric field. One suitable material is NuSil CF19-2186 as provided by NuSil Technology of Carpenteria, California. Other exemplary materials include silicone elastomers such as those provided by Dow Corning of Midland, Mich., acrylic elastomers such as VHB 4910 acrylic elastomer as produced by 3M Corporation of St. Paul, Minn., polyurethanes, thermoplastic elastomers, copolymers comprising PVDF, pressure-sensitive adhesives, fluoroelastomers, polymers comprising silicone and acrylic moieties, and the like. Polymers comprising silicone and acrylic moieties may include copolymers comprising silicone and acrylic moieties, polymer blends comprising a silicone elastomer and an acrylic elastomer, for example. Combinations of some of these materials may also be used as the electroactive polymer in transducers. The constrictive occluding transducer 104 may be coated with a suitable biomedical material to avoid rejection or other unfavorable interaction with the body. Biomedical materials are materials that are physiologically inert to avoid rejection or other negative inflammatory response. Polyester, polytetrafluoroethylene (PTFE), expanded PTFE (ePTFE) and polypropylene are examples of biomedical materials.

Deflection of electroactive polymer transducers includes linear expansion and compression in one or more directions, bending, and axial deflection when the polymer is rolled. As illustrated by comparing the length 212, width 210, and depth 208 dimensions of FIGS. 2A and 2B electroactive polymer transducers deflect in all dimensions simultaneously. When a voltage is applied to the electrodes of the transducer elements 122, 124 through wires 110, the radial thicknesses of the transducer elements decrease (as illustrated by element 122 in FIG. 1C), increasing the diameter of the inner aperture 114 and permitting fluid to flow in the duct. When the voltage is reduced, the radial thickness of the elements 122, 124 increases (as illustrated by the element 124 in FIG. 1C), decreasing the diameter of the inner aperture 114 and occluding fluid flow in the duct 103. An implantable power source and control unit 112 includes a source of electrical energy and a system for controlling the application of the energy to the constrictive occluding transducers 122, 124.

The size of the inner aperture 114 can be selected to encircle the outer surface of the duct to which the artificial sphincter 100 is to be applied. In addition, the size of the inner aperture 114 can be varied by altering the voltage applied to the occluding transducers 122, 124 so that the pressure exerted by the artificial sphincter 100 on the duct 103 can be minimized to promote blood flow and protect sensitive tissues. By providing feedback, a control can adaptively adjust the occluding transducers 122, 124 to respond to transient pressure pulses such as those generated in the bladder while minimizing the pressure on the duct. A piezoelectric load sensing transducer in the latch 107 can provide a feedback signal to enable adjustment of the occluding transducers 122, 124.

The artificial sphincter 100 illustrated in FIGS. 1A-1C includes a substantially rigid, circular case with a C-shaped cross-section to retain the two elements of the occluding transducer 104. However, the function of the case is to restrain the transducer proximate to the duct and to anchor the reaction of the actuated transducers. The case may comprise a flexible, relatively inelastic material that is attached to the transducer elements 122, 124 to restrain their position and mutually resist the reaction forces. For example, the case 102 of the artificial sphincter 100 may be a band of relatively inelastic biomedical material that is adhered to the occluding transducers and linked at the ends by a suture.

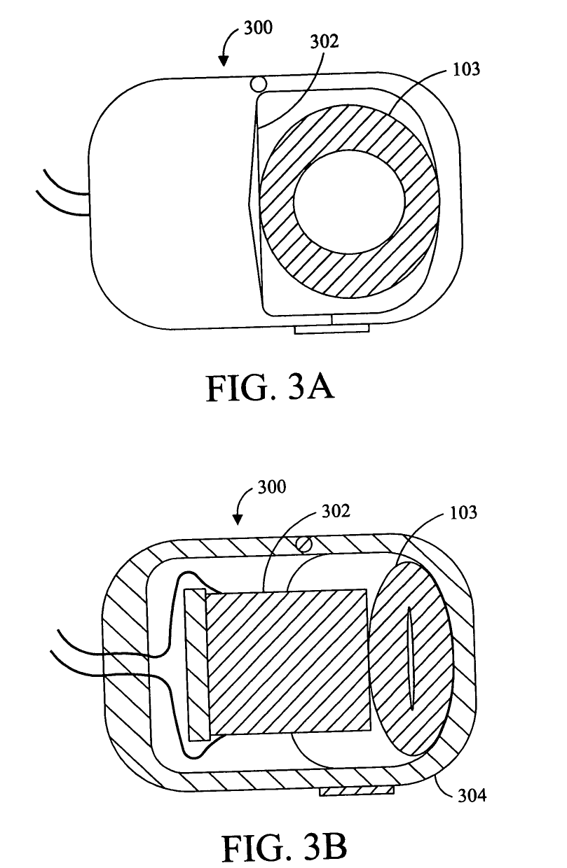

Referring to FIGS. 3A and 3B, in another embodiment of an exteriorly applied artificial sphincter 300, a single electroactive polymer transducer element 302 elongates and contracts in response to the application of electrical energy to displace a portion of the exterior surface of a duct 103 that is positioned between a surface of the transducer 302 and a portion of the case 304.

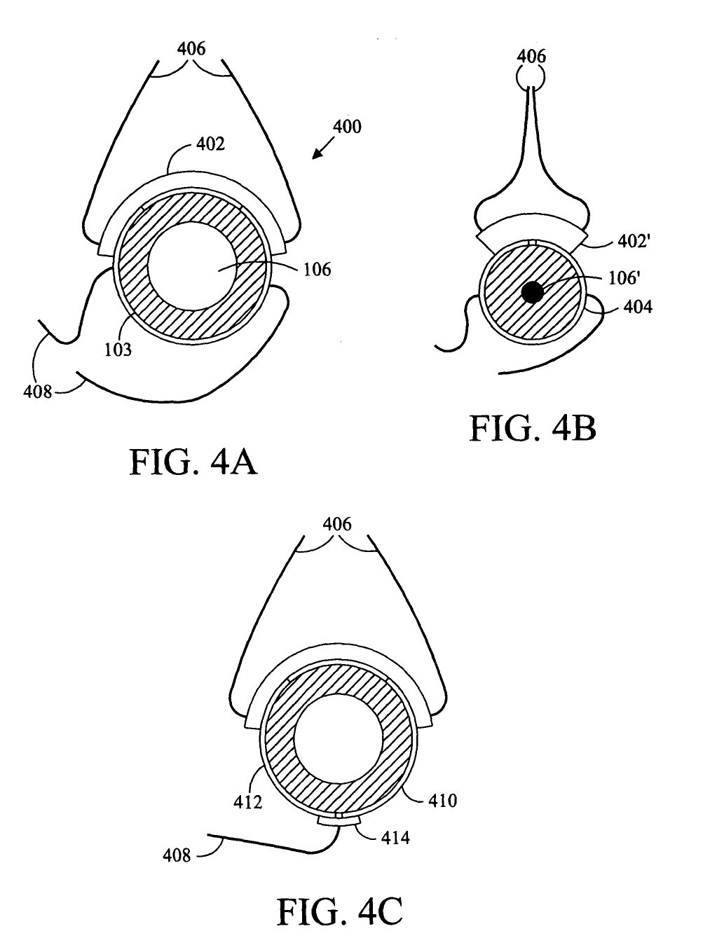

Referring to FIGS. 4A, 4B and 4C, another embodiment of an artificial sphincter 400 for duct exterior application comprises an electroactive occluding transducer 402 and a flexible case or band 404 that partially encircles the periphery of the duct 103. The occluding transducer 402 is attached to the two ends of the flexible case 404. When energized through wires 406, the transducer 402′ thickens and shortens drawing the ends of the case 404 together, shortening the circumference of the periphery of the portion of the wall under the band and collapsing the duct's aperture to block fluid flow. The case 404 may comprise a substantially inelastic biomedical material which may or may not be an electroactive polymer. If the case 404 is constructed of an electroactive polymer, the case can provide a feedback signal indicating the force being exerted by the artificial sphincter since the voltage at the electrodes of an electroactive polymer varies with the stress in the polymer. A control system sensing a feedback signal in wires 408, can adjust the tension in the case 404 by varying the electrical energy applied to the occluding transducer 402 so that the clamping pressure can be minimized to protect the tissues of the duct 103 and periodically reduced, momentarily, to promote circulation, but adaptively adjusted to compensate transient pressure pulses in the duct. Similarly, as illustrated in FIG. 4C, the case may be split into two elements 410, 412 connected by a piezoelectric load cell 414 that provides the feedback signal.

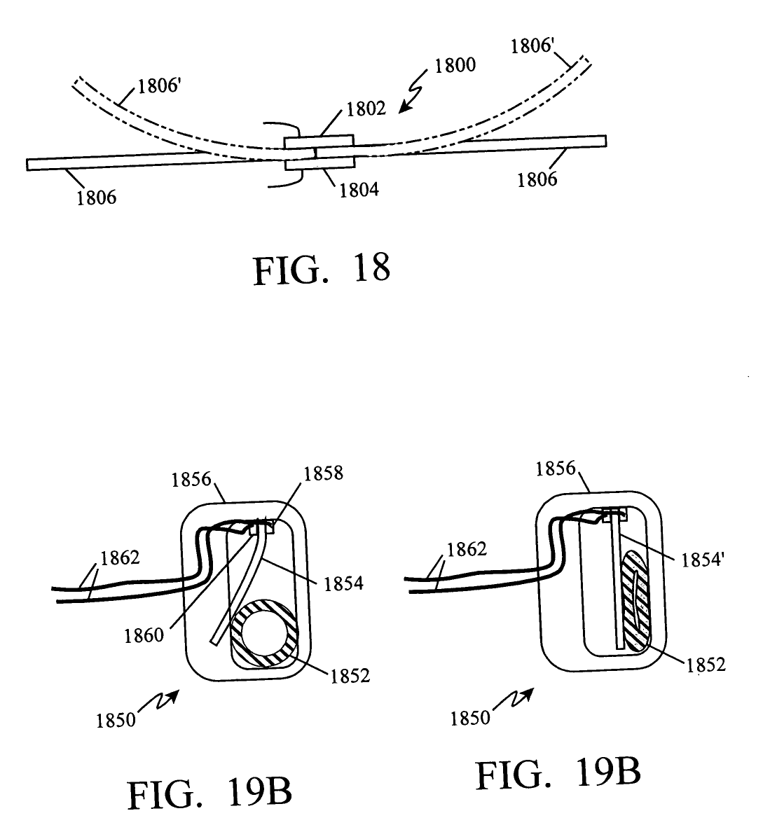

The occluding transducer may comprise a polymer-metal composite actuator. An ionic polymer-metal composite (IPMC) comprises a polymer having ion exchanging capability that is first chemically treated with an ionic salt solution of a conductive medium, such as a metal, and then chemically reduced. An ion exchange polymer refers to a polymer designed to selectively exchange ions of a single charge with its on incipient ions. Ion exchange polymers are typically polymers of fixed covalent ionic groups, such as perfluorinated alkenes, styrene-based, or divinylbenzene-based polymers. Referring to FIG. 18, a simple polymer-metal composite actuator 1800 comprises suitable electrodes 1802, 1804 attached to one or more polymer-metal composite elements 1806. When a time varying electric field is applied to the electrodes 1802, 1804 attached a polymer-metal composite element 1806, the element will exhibit a large dynamic deformation 1806′. Referring to FIGS. 19A and 19B, an embodiment of a duct occluding transducer 1850 incorporates a polymer metal composite transducer 1854 for displacing the exterior surface of a duct 1852. The transducer 1854 is restrained relative to the duct surface by a 1856. A voltage can be applied to the electrodes 1858, 1860 of the contractile transducer 1854 through wires 1862 causing the transducer 1854′ to deflect as illustrated in FIG. 19B, displacing the exterior surface and collapsing the duct 1852.

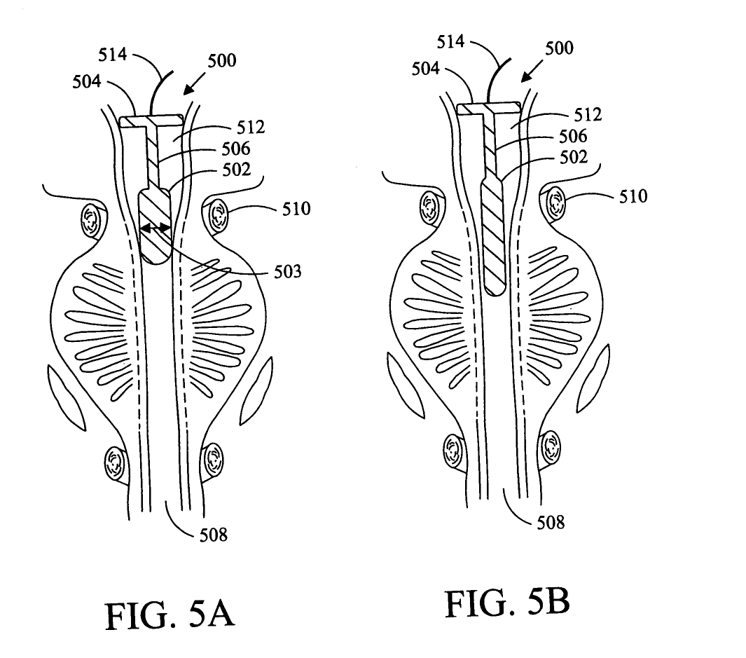

The fluid flow control device or artificial sphincter may comprise a transducer implantable interiorly in the aperture of the duct. An interiorly located artificial sphincter controls the flow of fluid in the duct by selectively occluding the aperture. The morbidity produced by devices that compress the duct from the outside is avoided with an internally implantable fluid control device and the device can often be implanted in the duct with minimal surgery. Referring to FIGS. 5A and 5B, in one embodiment the artificial sphincter 500 comprises generally a dilation element 502, an anchor 504, and a tether 506. The dilation element 502 comprises generally a cylinder of an electroactive polymer material that diametrically expands, as illustrated in FIG. 5A, or contracts, as illustrated in FIG. 5B, in response to the application of electrical energy. As the diameter 503 of the dilation element expands, the periphery of the dilation element makes contact with the wall of the duct blocking flow in the aperture 106. When the diameter 503 contracts, the periphery of the dilation element is reduced, creating a passage for fluid flow between the dilation element 502 and the inner surface of the duct wall. Preferably, the dilation element 502 is installed at the level of the duct's natural sphincter to enhance the function of the muscle in controlling fluid flow. For example, in treating urinary incontinence, the dilation element 502 is preferably installed in the urethra 508 at the level of the urethral sphincter 510, but the device can be installed at any position in the duct.

The position of the dilation element 502 in the duct is maintained by a tether 506 that connects the dilation element and to an anchor 504. The anchor 504 may be a shape known to be appropriate for the duct system. When used in the urethra 508, the artificial sphincter 500 is implantable with a cystoscope or other urethral instrument. Typically, the anchor 504 is foldable to permit insertion of the artificial sphincter 500 in the urethra 508. With the dilation element 502 of the artificial sphincter 500 in place, the anchor 504 projects into the bladder 512 at the end of the elongated tether 506. When unfolded, the anchor 504 is of sufficient size to prevent the artificial sphincter 500 from being expelled from the urethra 508.

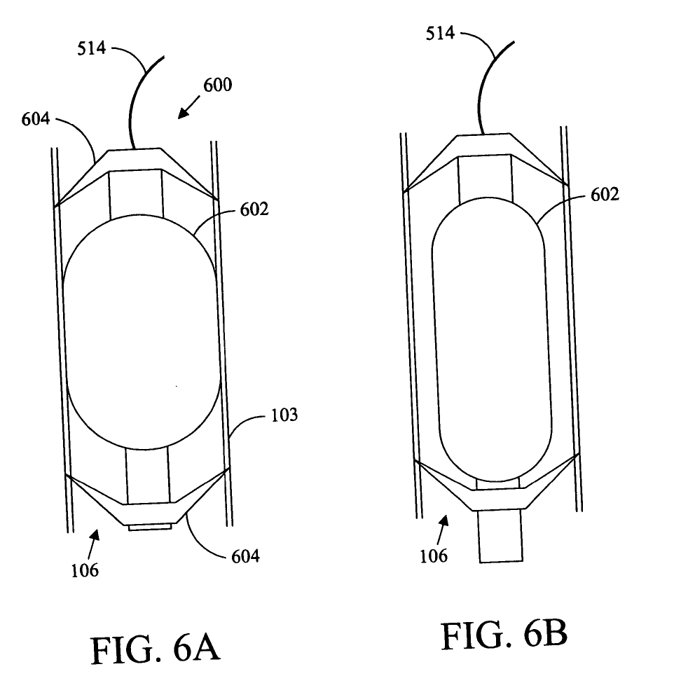

Similarly, a dilation element 602 of an interiorly implantable artificial sphincter 600 could be anchored in a duct 103 by extendable prongs 604 that implant in the wall of aperture 106 as illustrated in FIGS. 6A and 6B.

When a voltage is applied to the electroactive polymer dilation element 502, 602 through wires 514, the diameter of the substantially cylindrical dilation element contracts producing an annular fluid flow path between the dilation element and the wall of the duct 103. To block flow, the voltage to the electrodes of the electroactive polymer dilation element 502, 602 is reduced causing the cylinder's diameter to increase, plugging the aperture 106.

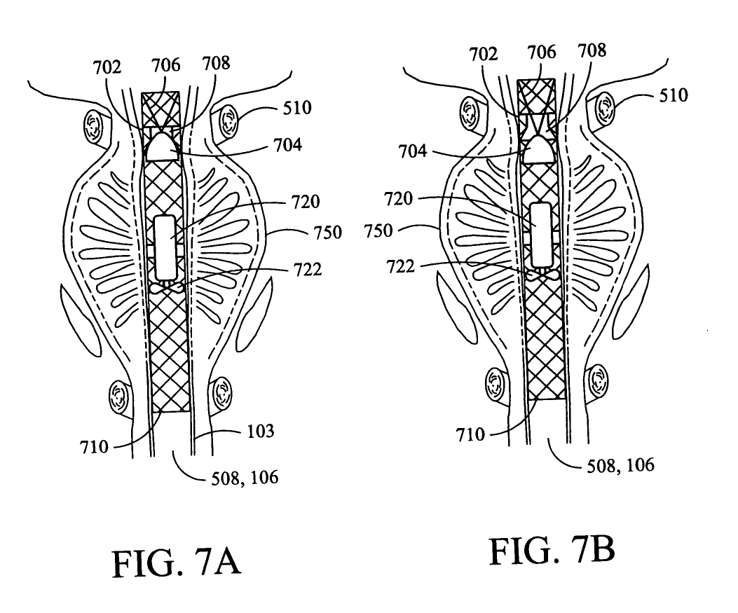

Another embodiment of the interiorly implantable artificial sphincter 700 is illustrated in FIGS. 7A and 7B. The artificial sphincter 700 comprises generally a valve seat 702, a complimentary valve spool 704, and a plurality of electroactive polymer actuators 706. The valve seat 702 fits closely in the aperture 106 of the duct so that fluid flowing in the duct flows through an aperture 708 in the valve seat. The valve seat 702 may be anchored in the duct 103 by a stent 710 or other known means. For example, benign prostate hypertrophy may be treated by inserting a stent into the urethra to support the urethra and resist occlusion of the urethra by the swollen prostate gland 750. A stent 710 typically comprises a metal or polymer tube that is collapsed for insertion into the fluid duct and then expanded to wedge in the walls of the duct. A portion of the stent 710 comprises the valve seat 702.

In the artificial sphincter 700, the valve spool 704 is restrained against the valve seat 702 by a plurality of electroactive polymer actuators 706 that are anchored to the stent 710. When the valve spool 704 is in contact with the valve seat 702, flow through aperture 708 of the valve seat is blocked. To enable flow, a voltage is applied to the electroactive polymer actuators 706 causing the actuators to lengthen and unseat the valve spool 702 as illustrated in FIG. 7B. As a result, fluid flow is enabled in the annular channel between the valve spool and the walls of the valve seat aperture 708. In addition, the valve spool 702 can comprise an electroactive polymer dilation that diametrically contracts when the valve is opened to increase the cross-section of the flow path and reduce flow losses in the artificial sphincter.

Piezoelectric and electrostrictive materials develop a polarized electric field when placed under stress or strain. Conversely, they undergo dimensional changes in an applied electric field. The dimensional change (i.e., expansion or contraction) of a piezoelectric or electrostrictive material is a function of the applied electric field. Piezoelectric and electrostrictive materials can possess a large number of combined and useful properties such as piezoelectric (electric field dependent strain), electrostrictive, dielectric, pyroelectric (temperature dependent polarization), ferroelectric (electric field dependent polarization) and electrooptic (electric field dependent optical birefringence).

Under an applied electric field, a piezoelectric crystal deforms along all its axes. It expands in some directions and contracts in others. The piezoelectric or strain coefficient describing this deformation is commonly denoted by the tensor dᵢⱼ:

dᵢⱼ=Xⱼ/Eⱼ(constant X)=Pᵢ/Xᵢ(constant E)

where x equals strain (extension per unit length); X equals stress (force per unit area); E equals electric field (volts per meter), and P equals polarization (Coulombs per square meter). The subscripts i,j refer to the crystal axes, or in the case of ceramics, to the direction of polarization of the ceramic. For example, dᵢⱼ is the strain coefficient in the lateral direction while d₃₃ is the strain coefficient for the longitudinal direction.

A typical ceramic device such as a direct mode actuator makes direct use of a change in the dimensions of the material, when activated, without amplification of the actual displacement. The direct mode actuator includes a piezoelectric or electrostrictive ceramic plate sandwiched between a pair of electrodes formed on its major surfaces. The device is generally formed of a material which has a sufficiently large piezoelectric and/or electrostrictive coefficient to produce the desired strain in the ceramic plate. By applying a voltage of appropriate amplitude and polarity between some dimensions of the device, it will cause the piezoelectric (or electrostrictive) material to contract or expand in that direction. When the device expands or contracts in one dimension (the thickness or longitudinal direction) it generally contracts or expands respectively, in dimensions in a plane perpendicular thereto (planar or transverse directions).

Direct mode actuators utilize either the longitudinal extensional mode or lateral extensional mode and are capable of sustaining high loads under compression but produce very little displacement (strain).

Indirect mode actuators achieve strain amplification via external structures. An example of an indirect mode actuator is a flextensional transducer. Flextensional transducers are composite structures composed of a piezoelectric ceramic element and a metallic shell, stressed plastic or fiberglass structure. The actuator movement of conventional flextensional devices commonly occurs as a result of expansion in the piezoelectric material which mechanically couples to an amplified contraction of the device in the transverse direction. In operation, they can exhibit up to about 0.5% strain at .+−0.25 V/mil applied electric field and can sustain loads up to several hundred pounds.

Indirect mode actuators include the unimorph, bimorph, multimorph and monomorph actuators. A typical unimorph is composed of a single piezoelectric element externally bonded to a flexible metal foil which is stimulated by the piezoelectric element when activated with a changing voltage and results in axial buckling or deflection as it opposes the movement of the piezoelectric element. The actuator movement for an unimorph can be by contraction or expansion.

A bimorph device typically includes an intermediate flexible metal foil sandwiched between two piezoelectric elements bonded to the plate. Electrodes are bonded to each of the major surfaces of the ceramic elements and the metal foil is bonded to the inner two electrodes. A multilayer device known as a multimorph can be made by stacking alternating layers of ceramic elements and metal plates. When a voltage is applied to the electrodes, the bimorph or multimorph bends or vibrates. Bimorphs and multimorphs exhibit more displacement than unimorphs because under the applied voltage, one ceramic element will contract while the other expands. Bimorphs and multimorphs can exhibit strains up to 20% at 25 V/mil.

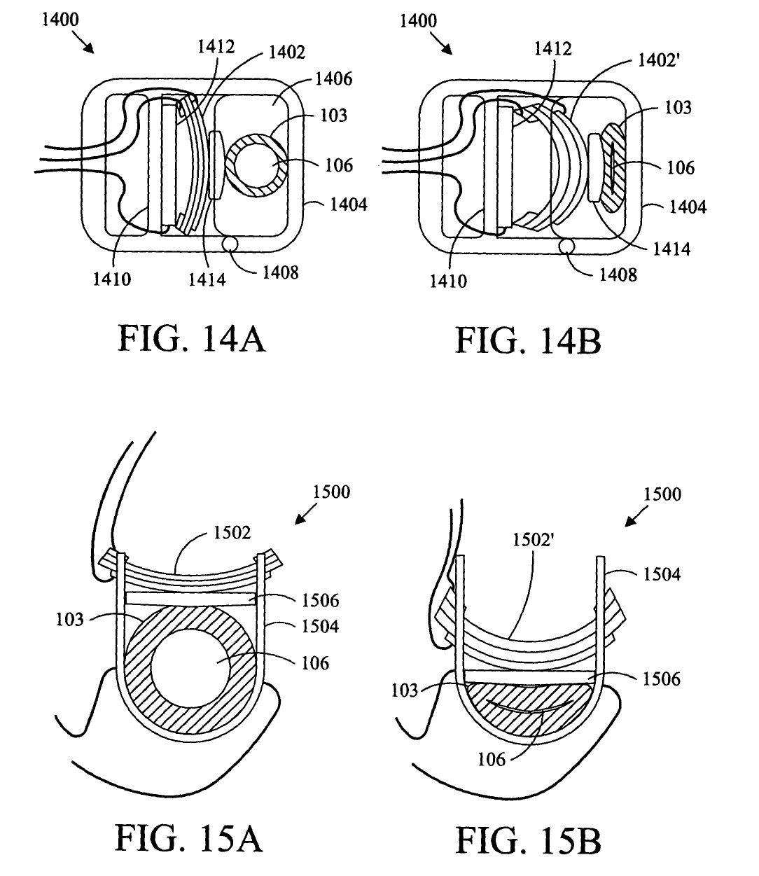

FIGS. 14A and 14B illustrated a flow control device or artificial sphincter 1400 having a piezoelectric bimorph duct occluding transducer 1402 that occludes the aperture 106 by displacing a portion of the exterior of the duct 103. The artificial sphincter 1400 comprises generally a case 1404 including an aperture 1406 through which the duct 103 passes. The case 1404 may include a hinge 1408 to permit the aperture 1406 to be opened to facilitate installation encircling the duct 103. The piezoelectric bimorph 1402 reacts against a piezoelectric force transducer 1412 that is trapped between the bimorph and a wall 1410 of the case 1404. The bimorph 1402′ presses a contact pad 1414 against the exterior of the duct 103 to occlude the duct's aperture 106. When energized, the bimorph 1402 deflects and the height dimension is reduced. This releases the pressure on the exterior of the duct 103 to enable flow in the duct.

Referring to FIGS. 15A and 15B, in another embodiment of an exteriorly applied fluid flow control device 1500, a piezoelectric bimorph 1502 is attached to the opposite ends a strap 1504 that partially encircles a duct 103. When the bimorph 1503′ bends the band 1504 is drawn against the reaction pad 1506 to occlude the aperture 106 of the duct 103. When the bimorph 1502 straightens, the pressure exerted by the band 1504 on the exterior of the duct 103 is relieved enabling flow in the duct. A portion of the band 1504 may comprise an electroactive polymer or a piezoelectric force transducer enabling a feedback signal relating the pressure being exerted by the band.

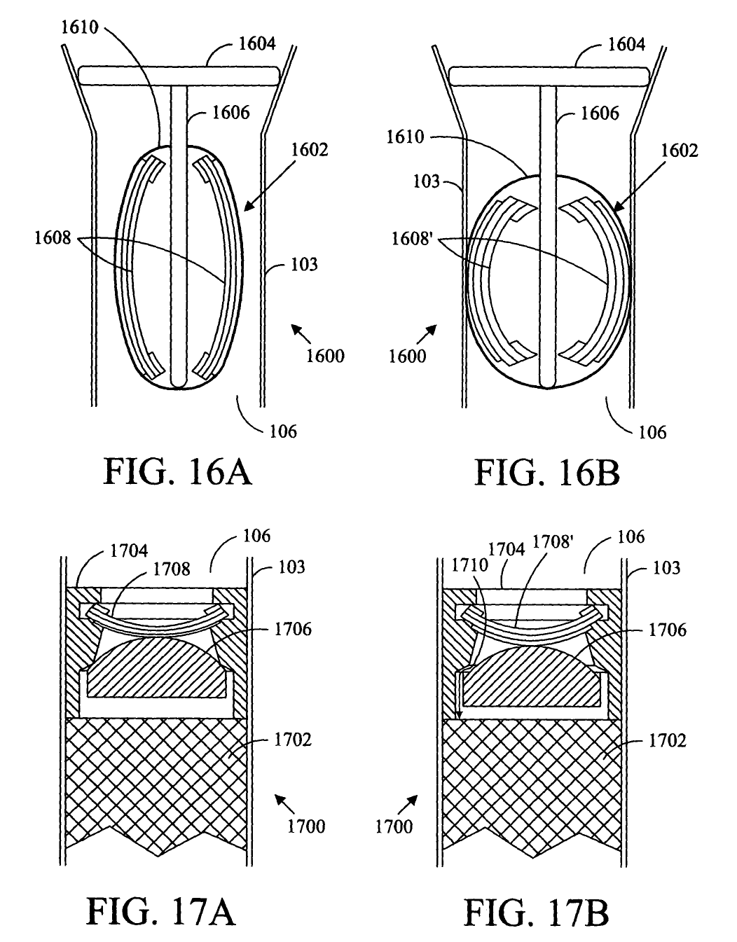

FIGS. 16A and 16B illustrate an embodiment of a fluid flow control device or artificial sphincter 1600 comprising a dilation element 1602 implanted interiorly in the aperture 106 of a duct 103. The dilation element 1602 is help in place by an anchor 1604 that wedges in the duct aperture 106 and is connected to the dilation element by a tether 1606. The dilation element 1602 comprises a plurality of piezoelectric bimorphs 1608 that react against the tether 1606 and are held in place by a flexible membrane 1610 of biomedical material. As illustrated in FIG. 16A, when the bimorphs 1608 are relatively straight, the diameter or cross section of the dilation element is less than that of the aperture 106 permitting fluid to flow. On the other hand, when the bimorphs 1608 are deflected, as illustrated in FIG. 16B, the diameter of the dilation element 1608 fills the duct aperture 106 to block fluid flow.

FIGS. 17A and 17B illustrated an additional embodiment of an artificial sphincter 1700 for implantation in a duct aperture 106 to control a flow of fluid. The artificial sphincter 1700 is anchored in the duct by connection to a stent 1702 that bears against the wall of the aperture 160 and comprises generally a valve seat 1704, a valve spool 1706, and a piezoelectric bimorph actuator 1708. When the bimorph actuator 1708 is relatively straight, as illustrated in FIG. 17A, the valve spool 1706 is held against the valve seat 1704 blocking the flow of fluid. When the bimorph 1708′ deflects, as illustrated in FIG. 17B, the valve spool 1706 is unseated from the valve seat 1704 permitting fluid 1710 to flow through the annular opening between the valve seat and spool.

Referring to FIG. 18, a polymer-metal composite transducer 1800 can be substituted for the piezoelectric transducers 1608 and 1708 of the artificial sphincters 1600 and 1700. Application of a time varying electrical field to the electrodes 1802, 1804 causes the polymer-metal composite element 1806 to deflect 1806′ producing duct occluding displacement in substantially the same manner as the piezoelectric tranducers 1608 and 1708. Referring to FIGS. 2A and 2B, an electroactive polymer transducer 200 could be used to actuate the valve spool 1706 of the artificial sphincter 1700 illustrated in FIG. 17.

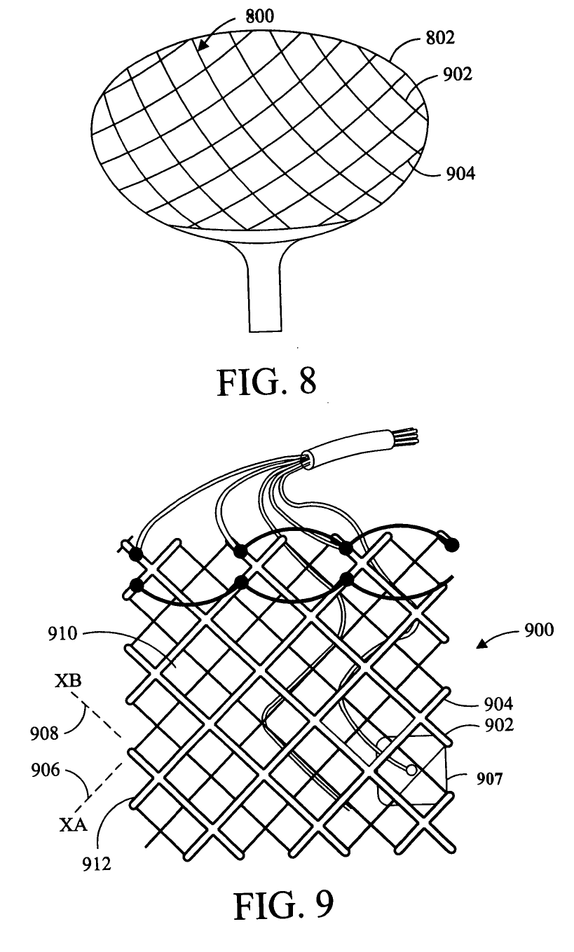

Urinary continence requires contemporaneous relaxation of the sphincter muscles of the urethra and contraction of the detrusor muscles in the bladder wall. When the detrusor contracts, the volume of the bladder is reduced, generating sufficient pressure at the inlet to overcome the pressure drop produced by flow restrictions in the urethra. If the detrusor muscles, or their associated nerves and fibers become inoperative because of disease or damage or if the flow restriction in the urethra becomes excessive, urinary retention (the inability to empty the bladder) results. If necessary to augment the body's capabilities, the fluid control system may include a fluid pressure assist device. Referring to FIG. 8, in the case of the urinary system, a first embodiment of the fluid pressure assist device comprises a constrictive jacket 800 fitted to and substantially encircling the exterior surface of the bladder 802 to compress the bladder during urination. The jacket 800 comprises a substantially spherical sack defining an internal volume which is sized to fit the distended bladder 802 while avoiding interference with the ureters, nervous input, and vascular supply to the bladder. As schematically illustrated in FIG. 9, the jacket 800 is a mesh material 900 comprising one or more filaments of an electroactive polymer. The mesh 900 comprises filamentary electroactive polymer contractile transducers 902 and 904 interwoven on a plurality of axes XA 906 and XB 908 defining a diamond-shaped open cell 910. On the other hand, filamentary transducers can be arranged along other axes to produce a mesh with triangular cells or cells of other shapes. In response to a voltage at the transducers'electrodes, the electroactive polymer contractile transducers 902, 904 either elongate or shorten. As the transducers 902, 904 shorten or lengthen, the volume of the jacket 800 is reduced or expanded, respectively, and the bladder is compressed to increase the intravesical pressure and aid the ejection of urine during urination or relaxed to permit the collection of urine expelled by the ureters. The fluid pressure assist device 800 can be fitted to the bladder and adjusted, post operatively, by permitting the contractile transducers 902, 904 to assume a length that produces an appropriate volume for the distended bladder. The pressure in the bladder 802 can be monitored by a pressure sensing transducer comprising a transducer 907, such as a piezoelectric pressure transducer, trapped between the jacket 800 and the bladder 802 or electroactive polymer filament 912 of the jacket mesh 900. A voltage at the electrodes of an electroactive polymer filament sensing transducer 912, which can be correlated to bladder pressure, will vary with the tension in the filament.

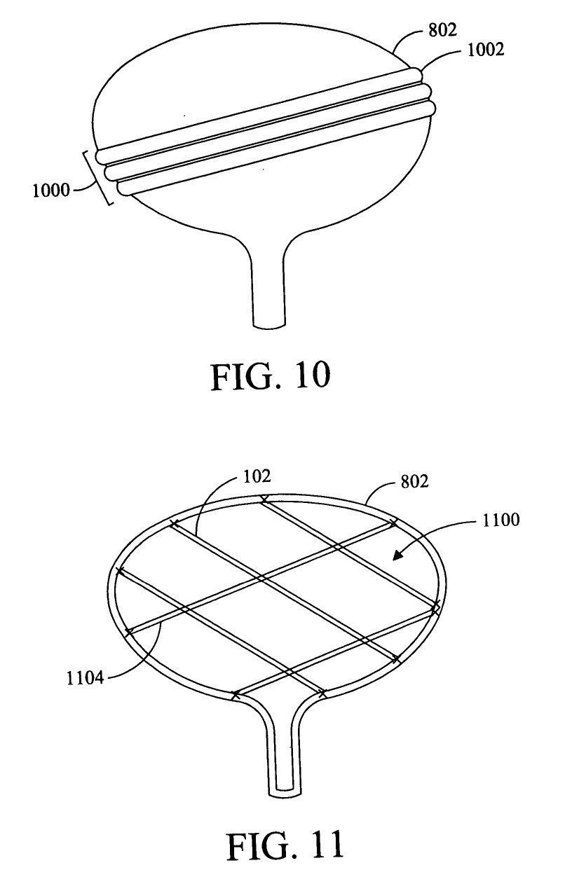

Referring to FIG. 10, in an alternative embodiment the contractile transducers 1002 of a fluid pressure assist device are incorporated into a girdle 1000 (indicated by a bracket) that encircles a surface of the bladder. The girdle 1000 can be retained on the surface of the bladder by a knit jacket or sock of biomedical material or by sutures or other suitable fasteners.

Referring to FIG. 11, in another embodiment the fluid pressure assist device 1100 is installed inside the bladder 802. Filaments of electroactive polymer 1102, 1104, which may be connected to each other, are attached to the interior of the bladder 802 walls by sutures 1106 or other suitable fastening methods. The application of electric energy to the electrodes of the electroactive polymer transducer filaments 1102, 1104 of the pressure assist device 1100 causes the filaments to contract, reducing the volume of the bladder 802.

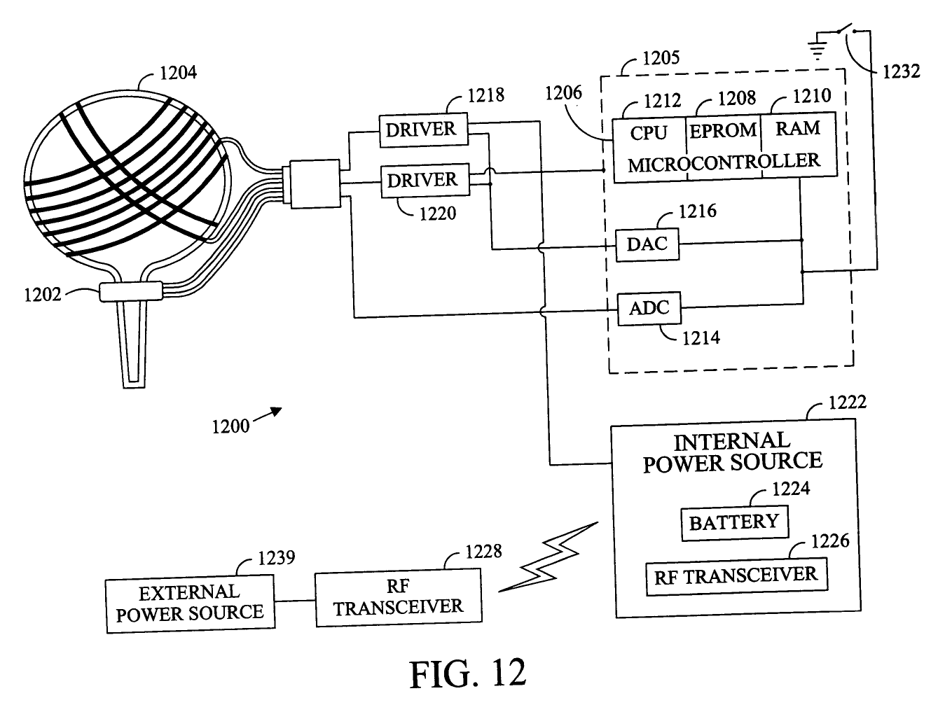

The operations of the fluid control devices are controlled and coordinated by a control system 1200, illustrated in block form in FIG. 12. For example, when the fluid control system is applied to relieve a dysfunction of the urinary system, flow through the urethra can be controlled by an artificial sphincter 1202 and, if necessary, pressure in the bladder can be increased by the pressure assist device 1204. Urinary continence requires occlusion of the urethra and relaxation of the bladder during the urine collection phase and coordinated relaxation of the urethral sphincter and contraction of the bladder wall during urination. In addition, particularly if an artificial sphincter is applied to the exterior of the area of the urethral sphincter, the force exerted on the urethra should be minimized to avoid morbidity of the urethral tissue, but may be momentarily increased to compensate for pressure transients in the bladder produced by a number of activities. The control system 1200 coordinates and controls the operation of the artificial sphincter 1202 and the pressure assist device 1204.

The control system 1200, comprises generally, a microcontroller 1206 including an erasable, programable, read-only memory (EPROM) 1208 to store program instructions used to relate system operating parameters, including requirements of a treatment regimen, user commands, and parameters sensed by sensing transducers of the fluid control system, and output signals directing operation of the actuating transducers controlling flow through the artificial sphincter 1202 and the fluid pressure assist device 1204; random access memory (RAM) 1210 to store data and program instructions during processing; and a central processor (CPU) 1212 to execute the program instructions and output signals directing action by the various transducers. The controller 1205 typically includes an analog-to-digital convertor (ADC) 1214 to convert analog signals output by the sensing transducers of the artificial sphincter 1202 and pressure assist device 1204 to digital data suitable for use by the microcontroller 1212, and a digital-to-analog convertor (DAC) 1216 to convert the digital output of the microcontroller to analog signals for operating drivers 1218, 1220 that control the application of electrical energy to the occluding and reservoir contracting transducers of the various fluid control devices.

Power for the elements of the fluid control system may be obtained from an internal power supply 1222 that may be included in the implantable power source and control unit 112. For example, since the transducers of the artificial urethral sphincter 1202 and the bladder pressure assist device 1204 are only energized four times daily, on average, the internal power supply may be implanted in the bladder. The internal power supply 1222 supplies electrical energy to the controller 1205 and to the device drivers 1218, 1220 which are connected by appropriate leads to the occluding transducers of the artificial sphincter 1202 and contractile transducers of the fluid pressure assist device 1204. Typically, the implantable internal power supply 1222 comprises a battery 1224 and, in some embodiments, a radio frequency transceiver 1226 receiving RF signals from an external radio frequency (“RF”) transceiver 1228. The external RF transceiver 1228 may recharge a battery 1224 within the internal power supply 1222 from an external power source 1230. The external RF transceiver 1228 may supply electrical power to the batteries 1224 through an inductive field coupling between the external RF transceiver and the internal power supply 1222. The technology for inductive field coupling, including electronic programming and power transmission through RF inductive coupling, has been developed and is employed in, for example, cardiac pacemakers, and automatic internal cardiac defibrillators.

The external RF transceiver 1228 may be used to transmit program instructions and data regarding electromechanical sensing and other system parameters to the controller 1205 which may also be housed in the implantable power source and control unit 112. The user can control the operation of the fluid control system by actuating a switch 1232 or by signals from the external RF transceiver 1228 to the internal RF transceiver 1226 which is coupled to the controller 1205.

The flow of fluid in the duct may also provide a source of energy for recharging the battery 1224 of the internal power supply. As illustrated in FIGS. 7A and 7B, a generator 720 driven by a fluid turbine 722 anchored in the urethral stent 710 can generate electrical energy for the battery 1224 from the flowing urine.

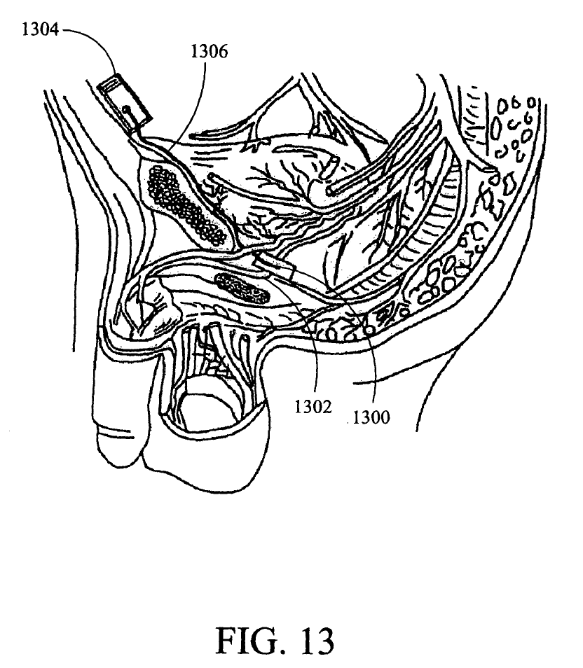

The fluid control system can be used to treat a number conditions that involve a dysfunction in the body's natural ability to control fluid. Referring to FIG. 13, for example, the fluid flow control system can be used to treat male impotence. An artificial sphincter 1300 is utilized to selectively obstruct the flow of blood from the superficial and/or deep dorsal veins 1302 of the penis, allowing erection due to the arterial engorgement of the corpus cavernosum penis. The actuation mechanism, under the control of a control unit 1304 similar to that described above in detail, can be remotely located from the artificial sphincter 1300 either within or outside of the body. The control unit 1304 may contain the microcontroller; interface circuitry, including ADC, DAC and drivers; and an energy source such as a lithium battery pack. This unit 1304 may be connected by control wires 1306 to the obturating sphincter 1300 on the veins.

Upon external activation by the user, the control unit 1304 begins a sequence of events that permits normal erection to occur. This external activation signal may be in the form of depressing a momentary contact switch located subcutaneously, a transcutaneous radio-frequency transmission from a manually activated transmitter, or a transcutaneous induced capacitance sensor that detects the presence of another person. The microcontroller 1205 activates an occluding transducer located in the artificial sphincter 1300 located around the superficial and deep dorsal veins 1302 to occlude the veins. The closure of most of the venous return from the penis allows full erection to occur as it does naturally. The control unit 1304 monitors for some secondary preprogrammed event to occur prior to releasing the occluding transducer to its quiescent position. This secondary event may be ejaculation, with an appropriate time delay, loss of proximity contact for greater than some preset time, secondary activation of the activation switch to cancel erection, or simply erection time exceeding a preset limit. The artificial sphincter 1300 used in this illustrated embodiment may be constructed substantially similar to several of the embodiments described above. This exemplary device 1300 and flow control system allows the restoration of normal penile function and acts as a permanent prosthetic device.

An implantable fluid flow control system comprising generally fluid flow control devices, such as an artificial sphincter and a fluid pressure assist device, and an associated control system, provides an effective and less traumatic method of treating a number of conditions involving dysfunction of the body's own fluid control systems.

The detailed description, above, sets forth numerous specific details to provide a thorough understanding of the present invention. However, those skilled in the art will appreciate that the present invention may be practiced without these specific details. In other instances, well known methods, procedures, components, and circuitry have not been described in detail to avoid obscuring the present invention.

All the references cited herein are incorporated by reference.

The terms and expressions that have been employed in the foregoing specification are used as terms of description and not of limitation, and there is no intention, in the use of such terms and expressions, of excluding equivalents of the features shown and described or portions thereof, it being recognized that the scope of the invention is defined and limited only by the claims that follow.

The invention claimed is:

1. A device implantable in a body for controlling a flow of fluid in a duct, said duct comprising a wall having an interior surface defining an aperture and an exterior, said implantable device comprising an electroactive polymer transducer operable to selectively occlude said aperture by displacement of said exterior of said wall of said duct.

2. The device of claim 1 wherein said electroactive polymer transducer operable to selectively occlude said aperture by displacement of said exterior of said wall of said duct comprises:
(a) an actuator having a dimension defined by a first surface and a second surface, said dimension being alterable by application of electrical energy; and
(b) a case having a first portion supporting said actuator proximate to said exterior of said wall on a first side of said duct and a second portion disposed proximate to said exterior of said wall on a second side of said duct, said second side of said duct being substantially diametrically opposed to said first side, such that a change in said dimension of said actuator will cause said first surface of said actuator to displace a portion of said wall in a direction substantially normal to said second side of said duct.

3. The device of claim 2 further comprising a sensing transducer outputting a signal responsive to a force exerted by said actuator.

4. The device of claim 1 wherein said electroactive polymer transducer operable to selectively occlude said aperture by displacement of said exterior of said wall of said duct comprises:
(a) a first actuator having a dimension defined by a first surface and a second surface, said dimension alterable by application of electrical energy, said actuator restrained such that said first surface is proximate said exterior of said wall of said duct;
(b) a second actuator having a dimension defined by a first surface and a second surface and alterable by application of electrical energy, said second actuator restrained such that said first surface is proximate said exterior of said wall and substantially diametrically opposed to said first surface of said first actuator; and
(c) a case to restrain said first and said second actuators relative to said duct and to react a force caused by a change in said dimension of said first and said second actuators.

5. The device of claim 4 further comprising a sensing transducer outputting a signal responsive to a force exerted by said actuator.

6. The device of claim 2 wherein said electroactive polymer transducer operable to selectively occlude said aperture by displacement of said exterior of said wall comprises:
(a) a band having a length defined by a first end and a second end, said band being arranged to encircle an arc of a periphery of said exterior of said wall, said arc having a length less than a circumference of said wall; and
(b) an actuator having a dimension alterable by application of electrical energy, said actuator being connected to said first end and said second end of said band such that a change in said dimension will reduce said periphery of said exterior wall.

7. The device of claim 6 further comprising a sensing transducer outputting a signal responsive to a force exerted by said actuator.

8. A device implantable in a body for controlling a flow of fluid in a duct, said duct comprising a wall having an interior surface defining an aperture and an exterior, said implantable device comprising a piezoelectric transducer operable to selectively occlude said aperture by displacement of said exterior of said wall of said duct.

9. The device of claim 8 wherein said piezoelectric transducer operable to selectively occlude said aperture by displacement of said exterior of said wall of said duct comprises:
(a) an actuator having a dimension defined by a first surface and a second surface, said dimension being alterable by application of electrical energy; and
(b) a case having a first portion supporting said actuator proximate to said exterior of said wall on a first side of said duct and a second portion disposed proximate to said exterior surface of said wall on a second side of said duct, said second side of said duct being substantially diametrically opposed to said first side, such that a change in said dimension of said actuator will cause said first surface of said actuator to displace a portion of said wall in a direction substantially normal to said second side of said duct.

10. The device of claim 9 further comprising a sensing transducer outputting a signal responsive to a force exerted by said actuator.

11. The device of claim 8 wherein said piezoelectric transducer operable to selectively occlude said aperture by displacement of said exterior of said wall of said duct comprises:
(a) a first actuator having a dimension defined by a first surface and a second surface, said dimension alterable by application of electrical energy, said actuator restrained such that said first surface is proximate said exterior of said wall of said duct;
(b) a second actuator having a dimension defined by a first surface and a second surface and alterable by application of electrical energy, said second actuator restrained such that said first surface is proximate said exterior of said wall and substantially diametrically opposed to said first surface of said first actuator; and
(c) a case to restrain said first and said second actuators relative to said duct and to react a force caused by a change in said dimension of said first and said second actuators.

12. The device of claim 11 further comprising a sensing transducer outputting a signal responsive to a force exerted by said actuator.

13. The device of claim 9 wherein said piezoelectric transducer operable to selectively occlude said aperture by displacement of said exterior of said wall comprises:
(a) a band having a length defined by a first end and a second end, said band being arranged to encircle an arc of a periphery of said exterior of said wall, said arc having a length less than a circumference of said wall; and
(b) an actuator having a dimension alterable by application of electrical energy, said actuator being connected to said first end and said second end of said band such that a change in said dimension will reduce said periphery of said exterior wall.

14. The device of claim 13 further comprising a sensing transducer outputting a signal responsive to a force exerted by said actuator.

15. A device implantable in a duct in a body for controlling a flow of fluid in said duct, said duct comprising a wall having an interior surface defining an aperture, said device comprising a transducer implantable in said aperture and operable to selectively occlude said aperture.

16. The device of claim 15 wherein said transducer implantable in said aperture comprises an electroactive polymer.

17. The device of claim 15 wherein said transducer implantable in said aperture comprises a piezoelectric material.

18. The device of claim 15 wherein said transducer implantable in said aperture comprises a polymer-metal composite.

19. The device of claim 15 wherein said transducer implantable in said aperture and operable to selectively occlude said aperture comprises a dilation element having a periphery responsive to electrical energy, said periphery being variable between a dilated periphery making substantial contact with said inner surface of said wall and a contracted periphery providing a space between said wall and said dilation element for fluid flow.

20. The device of claim 19 further comprising: an anchor connected to said dilation element to restrain said dilation element in said aperture.

21. The device of claim 19 wherein said transducer implantable in said aperture comprises an electroactive polymer.

22. The device of claim 19 wherein said transducer implantable in said aperture comprises a piezoelectric material.

23. The device of claim 15 wherein said transducer implantable in said aperture and operable to selectively occlude said aperture comprises:
(a) a valve seat restrained in said aperture, said valve seat comprising a channel for a flow of said fluid in said duct;
(b) a valve spool movable between a first position in contact with said valve seat to block said flow of said fluid in said channel and a second position not in contact with said valve seat to enable said flow; and
(c) an actuator responsive to electrical energy to move said valve spool from said first position to said second position.

24. The device of claim 23 wherein said actuator comprises an electroactive polymer.

25. The device of claim 23 wherein said actuator comprises a piezoelectric material.

26. The device of claim 23 wherein said transducer implantable in said aperture comprises a polymer-metal composite.

27. The device of claim 21 further comprising a stent engaging the interior of said aperture and connected to said valve seat.

28. The device of claim 21 wherein said valve spool movable between a first position in contact with said valve seat and a second position not in contact with said valve seat comprises an electroactive polymer dilation element having a dimension responsive to electrical energy, said dimension being variable between a dilated dimension substantially filling said channel and a contracted dimension smaller than a diameter of said channel.

29. A device implantable in a body for increasing a fluid pressure in a reservoir, said reservoir comprising a flexible wall having an exterior surface and an interior surface defining a volume, said device comprising an electroactive contractile transducer arranged to compress said exterior surface of said reservoir.

30. The device of claim 29 wherein said electroactive contractile transducer arranged to compress said exterior surface of said reservoir comprises a filament mesh including a filament comprising an electroactive polymer.

31. A device implantable in a body for increasing a fluid pressure in a reservoir, said reservoir comprising a flexible wall having an exterior surface and an interior surface defining a volume, said device comprising an electroactive contractile transducer having a first and a second end, said first and said second ends being attached to said inner surface of said wall and defining a length of said transducer, said length being alterable by application of electrical energy to said contractile transducer.

32. A system for controlling a flow of fluid in a body duct including a wall having an interior surface defining an aperture for fluid flow and an exterior, said system comprising
(a) an electroactive polymer duct occluding transducer operable to selectively occlude said duct in response to a first signal;
(b) a program executable by a data processing device including a program instruction directing an output of said first signal; and
(c) a data processing device outputting said first signal to said electroactive polymer duct occluding transducer in response to said program instruction.

33. The system of claim 32 further comprising:
(a) a sensing transducer outputting a second signal to said data processing device, said second signal being responsive to one of a fluid pressure and a force exerted by said electroactive polymer duct occluding transducer; and
(b) another program instruction executable by said data processing device relating said second signal to said first signal.

34. The system of claim 32 further comprising
(a) a fluid pressure assist device for increasing a pressure of said fluid in a reservoir having a flow connection to said duct, said fluid pressure assist device being response to third signal from said data processing device; and
(b) another program instruction executable by said data processing device relating said first signal and said third signal.

35. The system of claim 34 further comprising:
(a) a sensing transducer outputting a second signal to said data processing device, said second signal being responsive to one of a fluid pressure and a force exerted by said electroactive polymer duct occluding transducer; and
(b) an additional program instruction executable by said data processing device relating said second signal to at least one of said first signal and said third signal.

36. The system of claim 32 wherein said electroactive polymer duct occluding transducer operable to selectively occlude said duct in response to said first signal comprises an electroactive polymer actuator arranged proximate to said exterior of said duct and having a dimension responsive to said first signal to displace a portion of said exterior of said duct occluding said aperture.

37. The system of claim 32 wherein said electroactive polymer duct occluding transducer operable to selectively occlude said duct in response to said first signal comprises an electroactive polymer actuator arranged interiorly in said aperture and having a dimension responsive to said first signal to block said flow of fluid in said duct.

38. A system for controlling a flow of fluid in a body duct including a wall having an interior surface defining an aperture for fluid flow and an exterior, said system comprising
(a) a piezoelectric duct occluding transducer operable to selectively occlude said duct in response to a first signal;
(b) a program executable by a data processing device including a program instruction directing an output of said first signal; and
(c) a data processing device outputting said first signal to said piezoelectric duct occluding transducer in response to said program instruction.

39. The system of claim 38 further comprising:
(a) a sensing transducer outputting a second signal to said data processing device, said second signal being responsive to one of a fluid pressure and a force exerted by said piezoelectric duct occluding transducer; and
(b) another program instruction executable by said data processing device relating said second signal to said first signal.

40. The system of claim 38 further comprising
(a) a fluid pressure assist device for increasing a pressure of said fluid in a reservoir having a flow connection to said duct, said fluid pressure assist device being response to third signal from said data processing device; and
(b) another program instruction executable by said data processing device relating said first signal and said third signal.

41. The system of claim 40 further comprising:
(a) a sensing transducer outputting a second signal to said data processing device, said second signal being responsive to one of a fluid pressure and a force exerted by said piezoelectric duct occluding transducer; and
(b) an additional program instruction executable by said data processing device relating said second signal to at least one of said first signal and said third signal.

42. The system of claim 38 wherein said piezoelectric duct occluding transducer operable to selectively occlude said duct in response to said first signal comprises an piezoelectric actuator arranged proximate to said exterior of said duct and having a dimension responsive to said first signal to displace a portion of said exterior of said duct occluding said aperture.

43. The system of claim 38 wherein said piezoelectric duct occluding transducer operable to selectively occlude said duct in response to said first signal comprises a piezoelectric actuator arranged interiorly in said aperture and having a dimension responsive to said first signal to block said flow of fluid in said duct.

44. A device implantable in a body for controlling a flow of fluid in a duct, said duct comprising a wall having an interior surface defining an aperture and an exterior, said implantable device comprising a polymer-metal composite transducer operable to selectively occlude said aperture by displacement of said exterior of said wall of said duct.
